(19) United States
(12) Patent Application Publication
(10) Pub. No.: US 2015/0032013 A1
(43) Pub. Date: Jan. 29, 2015

(54) DISPOSABLE SHEATH

(71) Applicant: Welch Allyn, Inc., Skaneateles Falls, NY (US)

(72) Inventors: Thaddeus Wawro, Skaneateles Falls, NY (US); David Tufenkjian, Skaneateles Falls, NY (US)

(21) Appl. No.: 14/341,250

(22) Filed: Jul. 25, 2014

Related U.S. Application Data
(60) Provisional application No. 61/859,044, filed on Jul. 26, 2013.

(51) Int. Cl. A61B 5/022; A61B 17/135

(52) U.S. Cl. CPC A61B 5/02233; A61B 17/135; A61B 2017/00526
USPC 600/499; 606/202; 29/525.01

(57) ABSTRACT

A cuff system includes a flexible substantially tubular sheath having a distal end, a proximal end, a central longitudinal axis, and a central channel extending along the longitudinal axis. The system also includes a first guide disposed at the proximal end of the sheath. The first guide is configured to control insertion of a cuff connector into the central channel in a first direction substantially parallel to the longitudinal axis. The system further includes an orifice proximate the distal end of the sheath. The orifice is configured to enable fluid connection between a portion of the cuff connector and an inflatable cuff while a remainder of the cuff connector is disposed within the central channel.

### CROSS-REFERENCE TO RELATED APPLICATION

This application claims the benefit of U.S. Provisional Application No. 61/859,044, filed Jul. 26, 2013, the entire disclosure of which is incorporated herein by reference.

### TECHNICAL FIELD

This application is directed to systems and methods for monitoring a patient, and in particular, to systems and methods for protecting the patient from cross-contamination during patient monitoring.

### BACKGROUND

Traditional non-invasive blood pressure monitoring devices operate by inflating a blood pressure cuff to a pressure above a patient's systolic blood pressure. For example, many physicians obtain blood pressure readings using blood pressure devices, such as sphygmomanometers, that include one or more tubes connecting the cuff to an inflation and/or measurement device. Because the systolic pressure is usually not know prior to inflation, the cuff must be inflated to such a pressure to ensure that the patient's arterial blood flow is completely occluded. Once above systole, pressure data may be collected and the cuff may be slowly deflated to enable the flow of blood through the artery to resume. Pressure data may also be collected during inflation and/or deflation of the cuff, and the collected data may be used to determine hemodynamic parameters associated with the artery. Such parameters may include, for example, an average blood pressure of the patient.

Recently, advancements have been made to blood pressure cuffs and their associated connection systems to facilitate the use of disposable “single-use” cuffs or “single-patient” cuffs. While such cuffs assist in reducing cross-contamination between patients and/or clinicians, the tubing and/or connectors used for inflating and deflating the cuffs are typically still used on numerous patients. While cleaning such tubing and connectors can act to further reduce cross-contamination, the time, difficulty, and expense of routinely sanitizing such components can discourage such cleaning practices. Accordingly, there is a need for a solution to reduce cross-contamination related to tubing and connectors used for blood pressure measurement in a simple cost effective manner.

The systems and methods described herein are directed toward overcoming the difficulties described above.

### SUMMARY

In an exemplary embodiment of the present disclosure, a cuff system includes a flexible substantially tubular sheath having a distal end, a proximal end, a central longitudinal axis, and a central channel extending along the longitudinal axis. The system also includes a first guide disposed at the proximal end of the sheath. The first guide is configured to control insertion of a cuff connector into the central channel in a first direction substantially parallel to the longitudinal axis. The system further includes an orifice proximate the distal end of the sheath. The orifice is configured to enable fluid connection between a portion of the cuff connector and an inflatable cuff while a remainder of the cuff connector is disposed within the central channel.

In another exemplary embodiment of the present disclosure, a cuff system includes a sheath having a distal end, a proximal end, a central longitudinal axis, and a central channel extending from the distal end to the proximal end along the longitudinal axis. The system also includes a first guide disposed at the proximal end of the sheath. The first guide includes a passage configured to accept insertion of a cuff connector and to guide movement of the cuff connector through the first guide to the central channel in a first direction substantially parallel to the longitudinal axis. The system also includes an orifice proximate the distal end of the sheath. The orifice permits passage of a portion of the cuff connector therethrough in a second direction substantially orthogonal to the longitudinal axis.

In a further exemplary embodiment of the present disclosure, a method of monitoring a patient includes disposing an inflatable cuff about a limb of the patient, and inserting a cuff connector through a passage of a first guide, the first guide being disposed at a proximal end of a substantially tubular sheath and guiding movement of the cuff connector in a first direction substantially parallel to a longitudinal axis of the sheath. The method also includes moving the cuff connector substantially along the longitudinal axis, within a central channel of the sheath, from the proximal end toward a distal end of the sheath, and passing a portion of the cuff connector through an orifice of the sheath in a second direction substantially orthogonal to the first direction. The method further includes fluidly connecting the portion to the cuff while a remainder of the cuff connector is disposed within the channel.

In another exemplary embodiment of the present disclosure, a method of manufacturing a sheath includes connecting a top panel of the sheath to a bottom panel of the sheath, the top and bottom panel forming a central channel 44 extending along a longitudinal axis of the sheath. The method also includes providing a first guide proximate a proximal end of the sheath, the first guide including a front face outside of the central channel, a back face opposite the front face and facing the channel, and a passage extending from the front face to the back face. In such a method, the first guide is positioned such that the longitudinal axis extends substantially centrally through the passage. The method also includes providing a second guide proximate a distal end of the sheath, the second guide overlaying an orifice of one of the top and bottom panels.

### BRIEF DESCRIPTION OF THE DRAWINGS

FIG. 1 illustrates a system according to an exemplary embodiment of the present disclosure.

FIG. 2 illustrates an exemplary sheath associated with the system of FIG. 1.

FIG. 3 illustrates a portion of the sheath shown in FIG. 2.

FIG. 4 further illustrates a portion of the sheath shown in FIG. 2.

FIG. 5 illustrates another portion of the sheath shown in FIG. 2.

FIG. 6 illustrates the portion of the sheath shown in FIG. 5 according to another exemplary embodiment of the present disclosure.

FIG. 7 illustrates a plurality of sheaths according to an exemplary embodiment of the present disclosure.

FIG. 8 illustrates a sheath of the present disclosure in an exemplary tethered condition.

FIG. 9 further illustrates the sheath shown in FIG. 8.

FIG. 10 illustrates a flow chart describing an exemplary method of the present disclosure.

### DETAILED DESCRIPTION

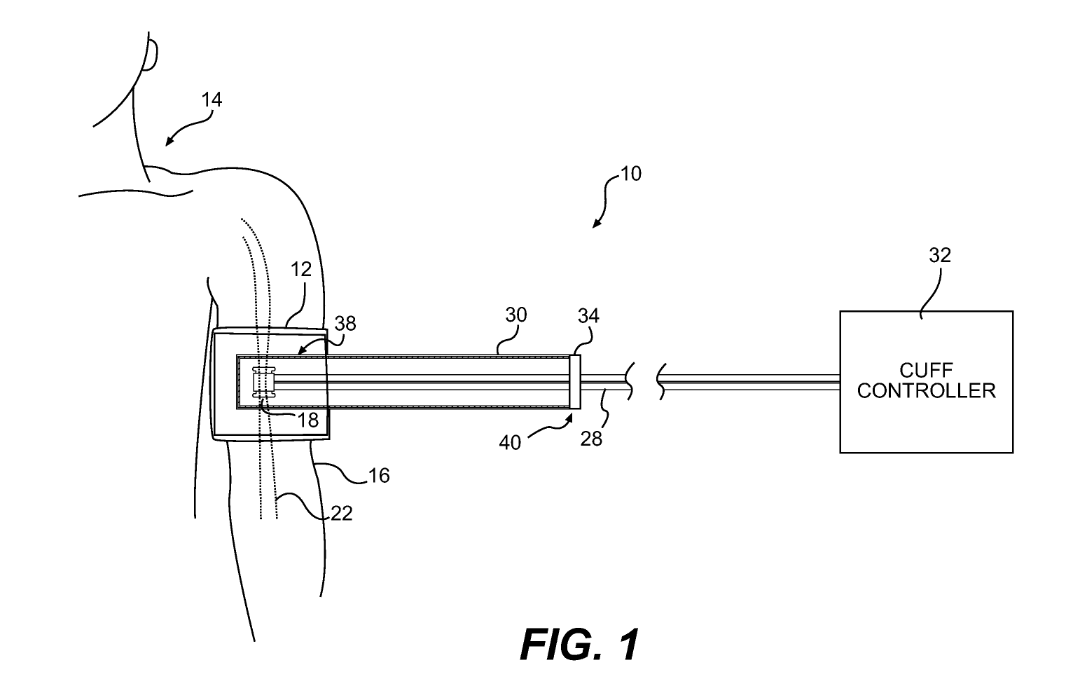

FIG. 1 illustrates a monitoring system 10 according to an exemplary embodiment of the present disclosure. The system 10 can be configured to monitor a patient 14, and in some embodiments, to determine a hemodynamic parameter of the patient 14. The system 10 can include a cuff 12 configured to at least to partially occlude the movement of blood through a vessel, vein, and/or artery 22 of the patient 14. In some embodiments, the cuff 12 can be configured to completely occlude the artery 22, and the artery 22 may be, for example, the brachial artery. For example, the cuff 12 may be inflated to any known occlusion pressure, and at such an occlusion pressure, the artery 22 may be at least partially occluded. The cuff 12 may also be deflated to a deflated pressure below (i.e., less than) the occlusion pressure, and at such a pressure, the artery 22 may be substantially unoccluded. Although shown in FIG. 1 as surrounding the upper arm of patient 14, the cuff 12 may be adapted for placement on or about any suitable limb 16 and/or other portion of the patient's body, including, for example, a wrist, a finger, an upper thigh, or an ankle. In addition, one or more cuffs 12 could be disposed at different locations about and/or on the patient 14 for use with the system 10.

The exemplary cuffs 12 of the present disclosure may be formed from any medically approved material known in the art. Such materials may be highly flexible, durable, and suitable for contact with, for example, the skin of the patient 14. Such materials may also be tear-resistant, fluid-impermeable, and recyclable. Such materials may include, for example, paper, cloth, mesh and/or polymers such as polypropylene or polyethylene. In still further exemplary embodiments, such materials may be coated and/or otherwise treated with one or more additives that cause the material to become biodegradable within a desired time interval (e.g., within 2 to 3 months). Each of the exemplary cuffs 12 described herein may have a respective length, width, and inflated height suitable for use with a particular patient 14. For example, a first cuff 12 intended to be used with an adolescent patient 14 may have a first deflated length and a first deflated width, and a second cuff 12 intended for use with an adult patient 14 may have a corresponding second deflated length and second deflated width. In such an exemplary embodiment, the first deflated length may be less than the second deflated length and the first deflated width may be less than the second deflated width. In exemplary embodiments, inflated lengths and widths of the exemplary cuffs 12 described herein may be different than the corresponding deflated lengths and widths.

The cuff 12 may include one or more bladders (not shown) or other like inflatable devices. Such a bladder may be formed from a single piece of material or, alternatively, from two or more pieces of material that are joined together through heat sealing, ultrasonic or RF welding, adhering, and/or other like processes. In still further exemplary embodiments, the cuff 12 may form one or more inflatable pockets that perform the same functions as a bladder. In such exemplary embodiments, the bladder may be omitted. It is understood that the cuff 12 and/or bladder may be inflatable to an occlusion pressure of approximately 160 mm Hg or greater to assist in at least partially occluding the artery 22.

In exemplary embodiments, the cuff 12 may include one or more ports (not shown) fluidly connected to the internal pocket or bladder to assist with inflation and/or deflation thereof. In exemplary embodiments, the port may comprise an open-ended substantially cylindrical structure, and a portion of the port may protrude from a top or outer surface of the cuff 12. Such an exemplary port may include, for example, a circumferential shelf, flange, ridge, shoulder, and/or other like structure to facilitate mechanical and/or fluid connection with one or more known fittings, adapters, and/or other like cuff connectors 18. For example, the port may be shaped, sized, and/or otherwise configured to mate with a corresponding cuff connector 18, and the cuff connector 18 may be fluidly connected to a bulb, pump, and/or other like cuff controller 32 utilized by a health care professional to inflate and/or deflate the cuff 12. Additional details concerning exemplary port designs are provided in co-owned U.S. Pat. No. 6,422,086, entitled “Low Profile Pressure Measuring Device,” the entire disclosure of which is incorporated herein by reference.

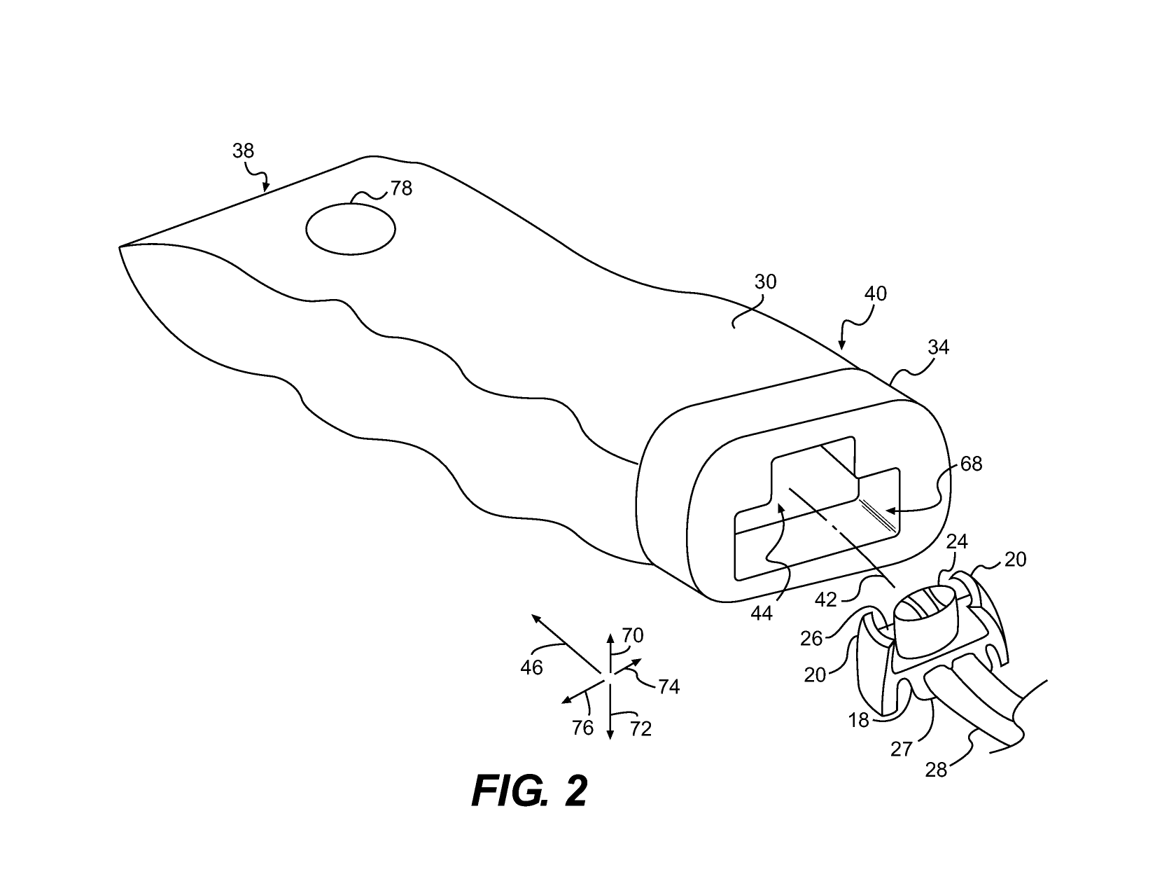

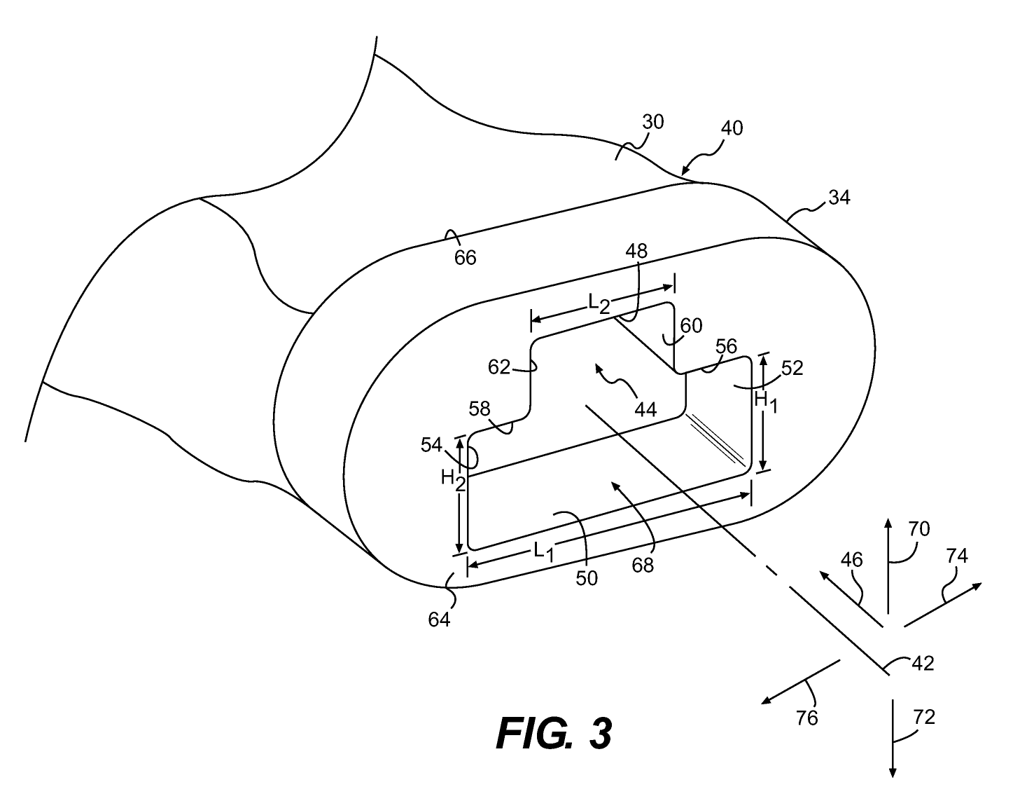

As will be described in greater detail below with respect to FIGS. 2 and 3, the cuff connector 18 may comprise any fitting or other like device configured to releasably connect to the port and to direct pressurized fluid to and/or from the cuff 12 via the port. The cuff connector 18 may be configured to releasably fluidly connect to the cuff 12 via the port, and may include one or more moveable, biased, spring-loaded, and/or otherwise adjustable components configured to facilitate a removable connection with the port. For example, the cuff connector 18 may include one or more arms, latches, hooks, prongs, snap connectors, and/or other like retention components 20 (FIG. 2) configured to engage the port, and to apply a retention force to the port. In such an embodiment, the retention component 20 may engage the port and may mechanically releasably connect the cuff connector 18 to the cuff 12. The cuff connector 18 may further include one or more O-rings, gaskets, seals, or other like components (not shown) to assist in forming a substantially fluid-tight connection with the port. In exemplary embodiments, the port may also include one or more O-rings, gaskets, seals, or other like components to assist in forming such a substantially fluid-tight connection with the cuff connector 18.

To further assist in forming a fluid connection with the cuff 12, the cuff connector 18 may include one or more portions extending therefrom and configured to mate with the port of the cuff 12. For example, such a portion may comprise a substantially hollow protrusion or other like extension 24. The extension 24 may include one or more passages configured to direct fluid from and/or to the cuff 12. In exemplary embodiments, the extension 24 may extend substantially perpendicularly from, for example, a top surface 26, a side surface, or a bottom surface 27 of the cuff connector 18. The extension 24 may be shaped, sized, positioned, and/or otherwise configured to accept a portion of the port therein when the cuff connector 18 is mechanically and/or fluidly connected to the cuff 12. Alternatively, the extension 24 may be shaped, sized, positioned, and/or otherwise configured to substantially surround the port, and may overlay the port when the cuff connector 18 is connected to the cuff 12.

With continued reference to FIG. 1, the pressure or volume of fluid within the cuff 12 may be controlled by the cuff controllers 32 fluidly connected and/or otherwise operably associated with the cuff 12 via the cuff connector 18. For example, the system 10 may include an automatic cuff controller, a manual cuff controller, and/or any other like cuff controller 32 known in the art. In such embodiments, the system 10 may further include one or more flexible hoses 28 fluidly connecting the cuff controller 32 and the cuff connector 18.

The various cuff controllers 32 of the present disclosure can include a pump or similar device configured to inflate and/or deflate the cuff 12. For example, an automatic cuff controller 32 could be controlled by a protocol or program stored in a memory associated with the cuff connector 32 to supply the cuff 12 with a fluid, such as air, to increase the pressure or volume within the cuff 12. Such an automatic cuff controller 32 may also be operatively connected and/or otherwise in communication with a cuff control module (not shown) of system 10. In such embodiments, the automatic cuff controller 32 may be configured to selectively inflate and deflate the cuff 12 in response to one or more control signals received from the cuff control module.

In additional exemplary embodiments, a manual cuff controller 32 may be configured to selectively inflate and deflate the cuff 12, and to thereby substantially occlude and unocclude the artery 22, in ways similar to the automatic cuff controller described above. However, a manual cuff controller 32 may be hand and/or otherwise manually operated by a user of the system 10 to inflate and deflate the cuff 12. A manual cuff controller 32 may comprise any manually operated device configured to supply fluid to and release fluid from the cuff 12. In exemplary embodiments, a manual cuff controller 32 may comprise a manually operated bulb, pump, or other like device commonly associated with conventional manual sphygmomanometers. In such embodiments, the manual cuff controller 32 may also include one or more valves (not shown) or other like flow control devices configured to maintain fluid, such as air, within the cuff 12 during inflation thereof, and to facilitate the gradual release of such fluid from cuff 12 during deflation. The valve may be manually controlled by the user to regulate the flow of air into and out of the cuff 12 during, for example, auscultation. In exemplary embodiments, the valve may comprise a manually controlled check valve or other like device.

In each of the exemplary embodiments described herein, the system 10 may be used to determine one or more characteristics associated with the artery 22 of patient 14. Such characteristics may include, for example, a systolic pressure, a diastolic pressure, a mean arterial pressure, and/or other known characteristics associated with the cuff 12, the artery 22, and/or the patient 14. It is understood that the system 10 may comprise any known oscillometric or auscultation system, and that the system 10 may be configured to perform and/or otherwise employ any known oscillometric or auscultation methods.

With continued reference to FIG. 1, the system 10 may also include one or more flexible, substantially tubular sheaths 30 configured to substantially enclose a portion of the hose 28 and/or the cuff connector 18 while the cuff connector 18 is mechanically and/or fluidly connected to the cuff 12. For example, the sheath 30 may permit direct mechanical and/or fluid connection between the cuff connector 18 and the port of the cuff 12 during use, while substantially enclosing a non-sterile portion of the hose 28 during use. In exemplary embodiments, the sheath 30 may comprise any medically acceptable material known in the art such as, for example, plastic, polymers, mesh, cloth, and/or other like materials. Such materials may include one or more of the materials discussed above with respect to the cuff 12. In such exemplary embodiments, the sheath 30 may form a substantially impervious barrier to contaminants such as air-borne pathogens, blood-borne pathogens, microorganisms, bacteria, and/or other contaminants commonly found in healthcare environments. In exemplary embodiments, the cuff 30 may be disposable and/or otherwise configured for single-use applications.

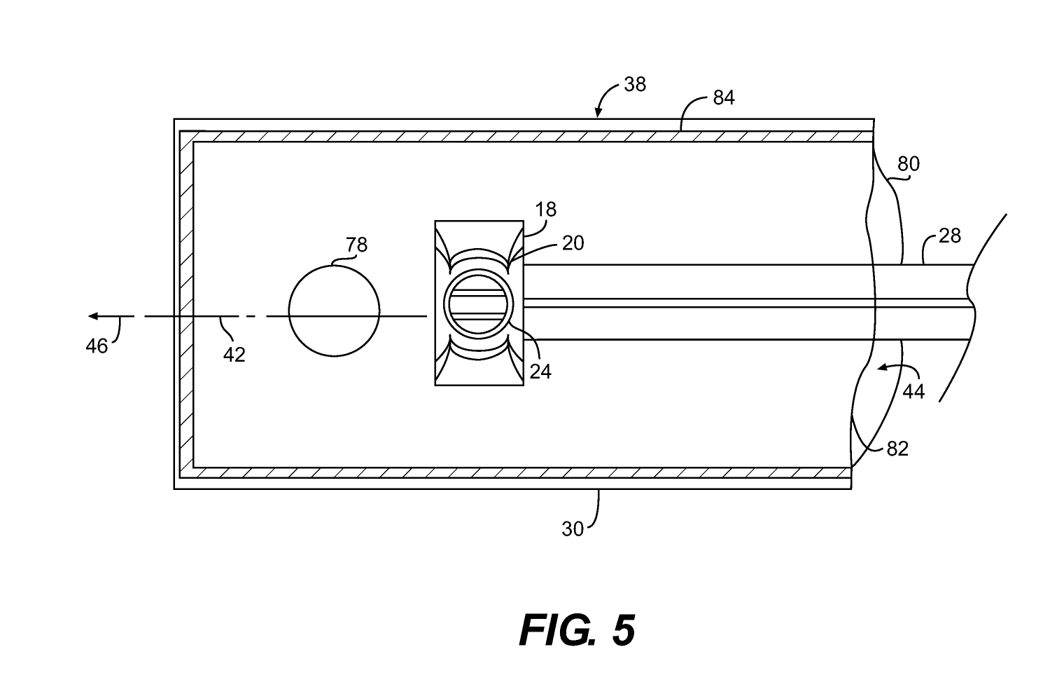

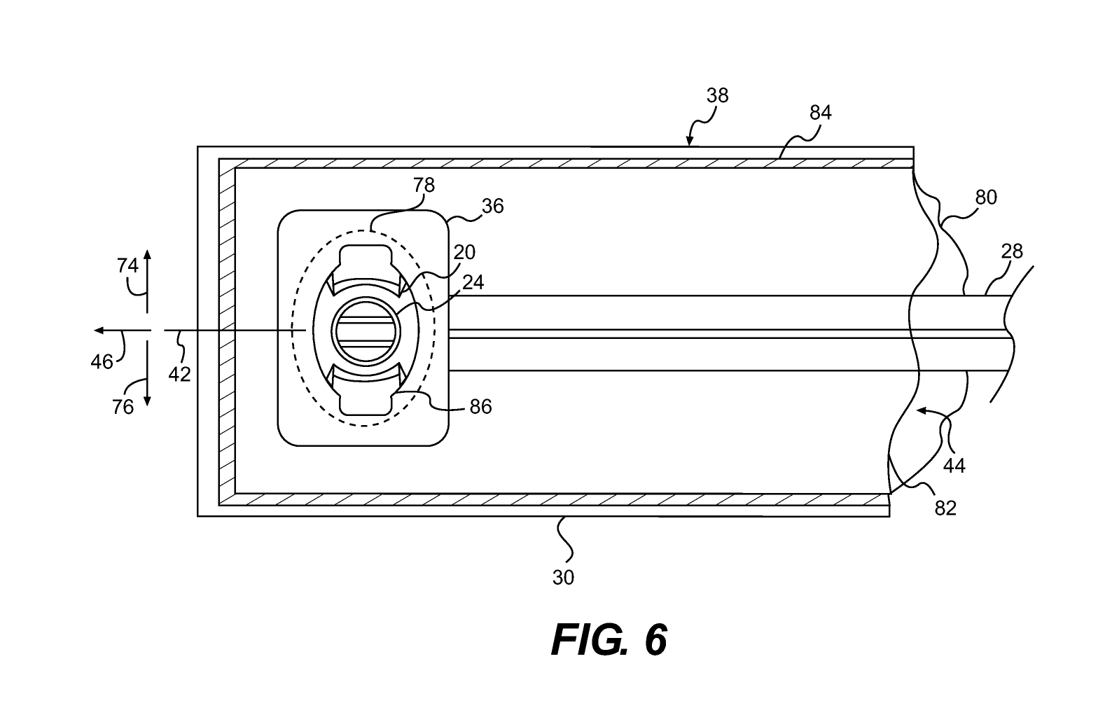

The sheath 30 may have a length that is substantially equal to a corresponding length of the hose 28. In such embodiments, the sheath 30 may extend from, for example, the cuff controller 32 to the cuff 12. In alternative exemplary embodiments, the sheath 30 may have a length less than a corresponding length of the hose 28. It is further understood that the sheath 30 may have a one-piece, two-piece, or multi-piece construction. For example, the sheath 30 may comprise a substantially planar top panel 80 (FIGS. 5 and 6) that is adhered to, sealed to, and/or otherwise connected to a corresponding substantially planar bottom panel 82 (FIGS. 5 and 6). An exemplary seal 84 connecting the top panel 80 with the bottom panel 82 is illustrated in FIGS. 5 and 6. Alternatively, in further exemplary embodiments, the sheath 30 may comprise a substantially hollow, substantially cylindrical one-piece construction.

In exemplary embodiments, the sheath 30 may include one or more guides configured to direct, control, and/or otherwise guide movement of the cuff connector 18 relative to the various portions of the sheath 30. For example, as shown in greater detail in at least FIGS. 2-6, the sheath 30 may include a distal end 38, a proximal end 40, a central longitudinal axis 42, and a central channel 44 extending along the longitudinal axis 42. The central channel 44 may be formed by and/or may otherwise extend between internal walls of the sheath 30. For example, in embodiments in which the sheath 30 includes a top panel 80 and a bottom panel 82, the central channel 44 may extend along and/or between the top panel 80 and the bottom panel 82. In alternative exemplary embodiments in which the sheath 30 comprises a substantially one-piece construction, the central channel 44 may extend along and/or between an inner circumference, inner diameter, and/or other inner surface of the sheath 30. In each of the exemplary embodiments described herein, the longitudinal axis 42 may extend substantially centrally through the central channel 44.

Further, a first guide 34 may be disposed at the proximal end 40 of the sheath 30, and the guide 34 may be configured to control insertion of the cuff connector 18 into the central channel 44 of the sheath 30 in a first direction that is substantially parallel to the longitudinal axis 42. Such an exemplary first direction as illustrated by the arrow 46 shown in FIG. 2. In each of the exemplary embodiments described herein, the guide 34 may be configured to control, direct, and/or otherwise guide movement of the cuff connector 18 in the first direction illustrated by arrow 46 as the cuff connector 18 is inserted into and/or moved through the guide 34. Additionally, the guide 34 may be configured to restrict movement of the cuff connector 18 in one or more directions relative to the first direction, and/or the longitudinal axis 42. For example, the guide 34 may be configured to restrict movement of the cuff connector 18 in a second direction substantially orthogonal to the longitudinal axis 42 and/or the first direction illustrated by arrow 46. In exemplary embodiments, such a substantially orthogonal second direction may comprise any direction extending radially from and substantially perpendicularly from the longitudinal axis 42 and/or the first direction illustrated by arrow 46.

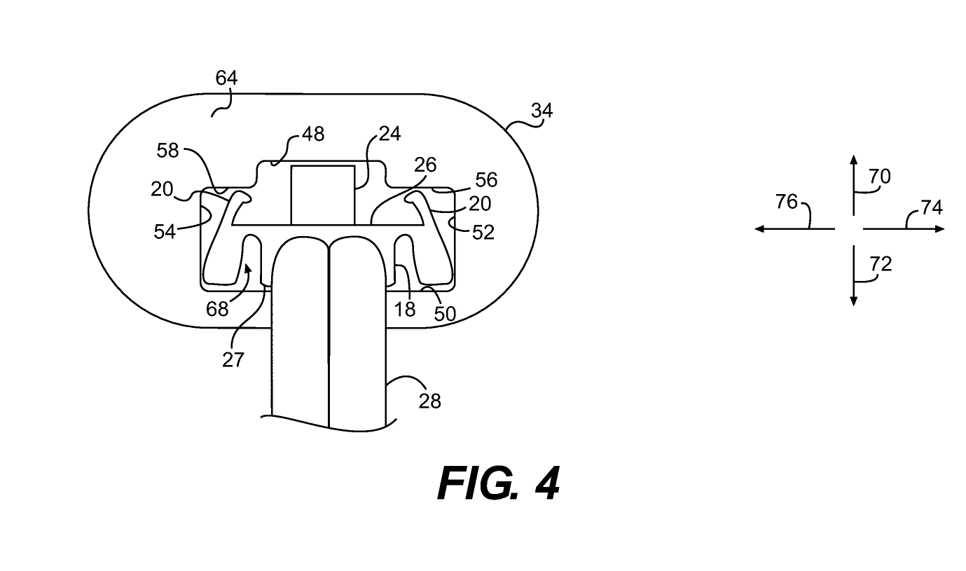

Such a substantially orthogonal second direction may be illustrated by one or more of arrows 70, 72, 74, and 76 shown in FIGS. 2-4. For example, as illustrated in FIG. 4, the guide 34 may be configured to restrict movement of the cuff connector 18 in at least one of the directions illustrated by arrows 70, 72, 74, and 76 while the cuff connector 18 is inserted into the guide 34 and passed therethrough into the central channel 44. In exemplary embodiments, due to the configuration of the guide 34, the cuff connector 18 may only be inserted into and passed through the guide 34 when the cuff connector 18 is positioned in a single unique orientation relative to the guide 34. Such an exemplary orientation is illustrated in at least FIGS. 2 and 4.

In exemplary embodiments, the guide 34 may include a passage 68 configured to accept insertion of the cuff connector 18, and to guide movement of the cuff connector 18 through the guide 34 to the central channel 44 in the first direction described above with respect to arrow 46. For example, as shown in at least FIG. 3, the guide 34 may include a front face 64 facing away from and/or otherwise disposed outside of the central channel 44. The guide 34 may also include a back face 66 opposite the front face 64. In such embodiments, the back face 66 may face and/or may otherwise be disposed within the central channel 44. Additionally, in exemplary embodiments the passage 68 may extend from the front face 64 to the back face 66. In further exemplary embodiments, the guide 34 may include at least one additional passage 68 configured to assist with guiding movement of the cuff connector 18 (and/or additional cuff connectors) through the guide 34. In such exemplary embodiments, each of the additional passages 68 may extend from the front face 64 to the back face 66. In exemplary embodiments, the longitudinal axis 42 may extend substantially centrally through at least one of the passages 68 described herein. In exemplary embodiments, the sheath 30 may be permanently connected to the guide 34, and in further exemplary embodiments, the sheath 30 may be removably connected to the guide 34. For example, to facilitate a removable connection between the sheath 30 and the guide 34, the sheath 30 may include one or more perforations, tear strips, pull cords, and/or other weakened sections (not shown) known in the art configured to facilitate simplified separation of the sheath 30 from the first guide 34 during or after use. Such simplified separation may assist in rapidly disposing of the sheath 30 and/or the first guide 34. Such simplified separation may also assist in recycling portions of the monitoring system 10 such as the sheath 30 and/or the first guide 34. In exemplary embodiments, the weakened section may be disposed proximate the back face 66 of the first guide 34 or along an outer surface of the first guide 34 extending from the front face 64 to the back face 66. Further, it is understood the weakened section may be constructed, positioned, and/or otherwise configured to avoid compromising the substantially impervious barrier against air-borne pathogens, blood-borne pathogens, bacteria, viruses, and/or other harmful contaminants formed by the sheath 30.

As shown in greater detail in FIGS. 2-4, the guide 34 may include one or more surfaces, or other like structures configured to assist in guiding movement of the cuff connector 18 through the passage 68. Such surfaces may comprise internal or external surfaces of the guide 34, and at least one such surface may be disposed adjacent to the front face 64, the back face 66, and/or the central channel 44. For example, one or more such surfaces may be at least partially formed by the front face 64 and/or the back face 66. Additionally, one or more such surfaces may form at least a portion of the passage 68, and may extend substantially from the front face 64 to the back face 66. Such surfaces may govern insertion of the cuff connector 18 into and passage of the cuff connector 18 through the guide 34 in a unique orientation such as, for example, the single unique orientation described above with respect to FIGS. 2 and 4. Further, while such surfaces may be described and/or illustrated herein as being substantially planar surfaces, in exemplary embodiments, such surfaces may be substantially contoured, substantially tapered, substantially curved, substantially rounded, and/or configured in any other way so as to assist in controlling insertion of the cuff connector 18 into the central channel 44, guiding movement of the cuff connector 18 through the guide 34 in the first direction, and/or restricting movement of the cuff connector 18 in one or more directions substantially orthogonal to the longitudinal axis 42. For example, one or more such surfaces may be tapered from the front face 64 to the back face 66 to assist a user with inserting the cuff connector 18 into the passage 68 via the front face 64.

In exemplary embodiments, one or more such surfaces may include a top wall 48, and a bottom wall 50 disposed opposite and substantially parallel to the top wall 48. As shown in at least FIG. 3, at least one of the top wall 48 and the bottom wall 50 may comprise a substantially planar surface extending substantially parallel to the longitudinal axis 42. For example, the top wall 48 and the bottom wall 50 may extend substantially along the longitudinal axis 42 in the first direction illustrated by arrow 46. Accordingly, the top and bottom walls 48, 50 may be configured to guide movement of the cuff connector 18 in the first direction as the cuff connector 18 is inserted into the passage 68. The guide 34 may also include a first sidewall 52, and a second sidewall 54 opposite and substantially parallel to the first sidewall 52. In exemplary embodiments, the first and second sidewalls 52, 54 may extend substantially perpendicularly from the bottom wall 50, and may be configured to further assist in guiding movement of the cuff connector 18 in the first direction as the cuff connector 18 is inserted into the passage 68.

As shown in at least FIG. 3, the guide 34 may also include a first shoulder 56 extending substantially perpendicularly from the first sidewall 52, and a second shoulder 58 extending substantially perpendicularly from the second sidewall 54. In such exemplary embodiments, at least one of the first and second shoulders 56, 58 may extend substantially parallel to the bottom wall 50 and/or the top wall 48. The guide 34 may also include a third sidewall 60 extending substantially perpendicularly from the first shoulder 56 and from the top wall 48. The guide 34 may further include a fourth sidewall 62 extending substantially perpendicularly from the second shoulder 58 and from the top wall 48. In such embodiments, the third and fourth sidewalls 60, 62 may extend substantially parallel to, for example, at least one of the first sidewall 52 and the second sidewall 54. It is understood that one or more of the top and bottom walls 48, 50, the first, second, third, and fourth sidewalls 52, 54, 60, 62, and/or the first and second shoulders 56, 58 may form a respective portion, section, and/or component of the passage 68.

Moreover, one or more of the top and bottom walls 48, 50, the first, second, third, and fourth sidewalls 52, 54, 60, 62, and/or the first and second shoulders 56, 58 may be shaped, sized, positioned, and/or otherwise configured to assist in guiding movement of the cuff connector 18 in the first direction described herein with respect to arrow 46, while restricting movement of the cuff connector 18 in one or more of the second directions described herein with respect to arrows 70, 72, 74, and 76. For example, these surfaces of the guide 34 may have respective lengths, widths, heights, and/or other configurations allowing for a substantially close fit between the cuff connector 18 and the surfaces of the passage 68 as the cuff connector 18 is inserted therein. Such a substantially close fit is illustrated in FIG. 4.

In exemplary embodiments, such a substantially close fit may be characterized by actual physical contact between the cuff connector 18 and one or more of the surfaces described herein while the cuff connector 18 passes through the passage 68. In other exemplary embodiments, however, one or more surfaces of the passage 68 may be positioned, dimensioned, and/or otherwise configured such that the cuff connector 18 may be passed through the passage 68 without physically contacting such surfaces. In such exemplary embodiments the surfaces of the passage 68 may be formed with relatively tight tolerances such that the cuff connector 18 may be spaced several tenths of an inch from at least one of the surfaces while passing through the passage 68.

For example, the bottom wall 50 may include a length L₁, and the top wall 48 may include a corresponding length L₂ that is less than L₁. Such lengths L₁, L₂ may be substantially equal to corresponding dimensions of the cuff connector 18, such as the respective dimensions of the extension 24 and the bottom surface 27, to facilitate the substantially close fit described above.

Moreover, as illustrated in FIG. 3, the first sidewall 52 may include a height H₁, and the second sidewall 54 may include a corresponding height H₂ that is substantially equal to the height H₁ of the first sidewall 52. Such heights may be substantially equal to corresponding heights of the cuff connector 18, such as, for example, a corresponding height of a respective retention component 20. The third and fourth sidewalls 60, 62 may also include respective heights similar to those described with respect to the first and second sidewalls 52, 54. It is understood that the respective heights of the third and fourth sidewalls 60, 62 may, for example, be chosen to correspond to an overall height of the cuff connector 18 and/or a height of the extension 24.

Thus, as illustrated in FIG. 4, one or more surfaces of the passage 68 may be positioned, dimensioned, and/or otherwise configured such that the portion 24, retention components 20, top surface 26, bottom surface 27, and/or other portions or components of the cuff connector 18 may pass in intimate contact with the surfaces of the passage 68 as the cuff connector 18 is passed through the passage 68. In exemplary embodiments, such intimate contact may result in a sliding and/or frictional engagement between the cuff connector 18 and the surfaces of the passage 68. For example, as the cuff connector 18 is passed through the passage 68, the portion 24 may slide along and/or may be guided by the top wall 48 while the bottom surface 27 and/or at least one of the retention components 20 may slide along and/or may be guided by the bottom wall 50. In such embodiments, at least one of the retention components 20 may slide along and/or may be guided by a corresponding one of the sidewalls 52, 54 as the cuff connector 18 is passed through the passage 68. In this way, the sliding and/or frictional engagement between the cuff connector 18 and the one or more surfaces of the passage 68 may guide movement of the cuff connector 18 in the first direction associated with arrow 46, and may restrict movement of the cuff connector 18 in one or more of the second directions associated with arrows 70, 72, 74, and 76. Such engagement may also ensure that the cuff connector 18 may only be inserted into and passed through the guide 34 when the cuff connector 18 is positioned in a single unique orientation relative to the guide 34.

As shown in at least FIGS. 2 and 5, the sheath 30 may also include at least one orifice 78 disposed proximate the distal end 38. In such exemplary embodiments, the orifice 78 may be configured to enable fluid connection between a portion of the cuff connector 18 and the cuff 12 while a remainder of the cuff connector 18 remains disposed within the central channel 44. For example, the orifice 78 may allow the extension 24 to pass therethrough such that a mechanical and/or fluid connection may be made between the cuff connector 18 and the one or more ports of the cuff 12 described above. As shown in FIG. 5, in an exemplary embodiment, the orifice 78 may be formed by the bottom panel 82 of the sheath 30. Alternatively, in additional exemplary embodiments, the orifice 78 may be formed by the top panel 80, and in further exemplary embodiments in which the sheath 30 comprises a one-piece construction, the orifice 78 may be formed by a substantially cylindrical wall of the sheath 30. In exemplary embodiments, the orifice 78 may comprise a through hole extending completely through the wall and/or panel 80, 82 of the sheath 30 in order to facilitate communication between the cuff connector 18 and the cuff 12. As shown in FIG. 5, the orifice 78 may be substantially circular so as to generally match the shape, size, and/or other configurations of the extension 24. Alternatively, the orifice 78 may be substantially elliptical, substantially square, substantially rectangular, substantially oblong, and/or any other shape, size, and/or configuration to enable passage of at least a portion of the extension 24 therethrough. The orifice 78 may be disposed at any convenient location on the sheath 30 to facilitate a desired connection between the cuff connector 18 and the cuff 12, and as illustrated in FIG. 5, in exemplary embodiments, the orifice 78 may be disposed substantially centrally along the longitudinal axis 42. It is understood that FIG. 5 illustrates an exemplary embodiment of the sheath 30 in which the second guide 36 has been omitted.

As shown in FIG. 6, on the other hand, in exemplary embodiments the guide 36 may be positioned at the distal end 38 so as to substantially overlay the orifice 78. In an exemplary embodiment, the guide 36 may be positioned on an outer surface of the sheath 30, and in further exemplary embodiments, the guide 36 may be positioned on an inner surface of the sheath 30. For example, in embodiments in which the orifice 78 shown in FIG. 5 is defined by the bottom panel 82 of the sheath 30, the guide 36 may be bonded, sealed, adhered, and/or otherwise connected to either an outer surface or an inner surface of the bottom panel 82 at a location that substantially corresponds to the orifice 78. Alternatively, in exemplary embodiments in which the sheath 30 comprises a one-piece construction, the guide 36 may be bonded, sealed, adhered, and/or otherwise connected to either an outer surface or an inner surface of the one-piece sheath 30. The guide 36 may be substantially planar, and may be formed from any of the materials described above with respect to the guide 34 and/or the sheath 30.

As shown in FIG. 6, the guide 36 may comprise one or more passages 86 configured to allow passage of the extension 24 therethrough. The passage 86 may have any shape, size, and/or other configuration to allow passage of the extension 24 therethrough while other portions of the cuff connector 18 remain disposed within the central channel 44. In exemplary embodiments, the guide 36 may include a substantially planar surface defining the passage 86, and the passage 86 may overlay the orifice 78 of the sheath 30. The passage 86 may be commensurate in size with, for example, a diameter, circumference, and/or other dimension of the orifice 78. For example, at least a portion of the passage 86 may have a diameter, circumference, and/or other dimension that is substantially equal to a corresponding dimension of the orifice 78. In alternative exemplary embodiments, a portion of the passage 86 may have a diameter, circumference, and/or other dimension that is less than a corresponding dimension of the orifice 78. For example, the sheath 30 illustrated in FIG. 6 comprises a guide 36 disposed on an outer surface of the bottom panel 82, and having an elliptical passage 86 including a portion having a diameter that is less than a corresponding diameter of the orifice 78. The passage 86 may also be substantially oblong and/or include one or more cutouts to facilitate passage of at least a portion of a retention component 20 therethrough. Passage of one or more retention components 20 through the passage 86 may further assist in connecting the cuff connector 18 to the cuff 12.

As described above with respect to the passage 68 of the first guide 34, the passage 86 of the second guide 36 may be configured to control and/or otherwise guide movement of the cuff connector 18 relative to the sheath 30. In particular, the guide 36 may be configured to guide movement of at least the extension 24 of the cuff connector 18 through the orifice 78. In such exemplary embodiments, the guide 36 may be configured to guide movement of the extension 24 through the orifice 78 in at least one of the second directions described above with respect to arrows 70, 72, 74, and 76. For example, the guide 36 may permit and/or otherwise control movement of the extension 24 in a direction substantially orthogonal to the longitudinal axis 42 and/or the first direction described above with regard to arrow 46. Such permitted movement may be in the direction of one of, for example, arrows 70, 72 (FIGS. 2-4). The guide 36 may also restrict movement of the extension 24 in the first direction associated with arrow 46, and in one or more directions substantially perpendicular to the first direction. For example, the guide 36 may restrict movement of the extension 24 in a direction associated with arrows 74, 76. In particular, as noted above with respect to the top wall 48, bottom wall 50, sidewalls 52, 54, 60, 62, and/or shoulders 56, 58 of the first guide 34, an inner circumference, inner diameter, and/or other inner surface of the portion of the guide 36 forming passage 86 may slidingly engage with the extension 24, and/or otherwise form a substantially close fit therewith, to assist in controlling or restricting movement of the extension 24 as described above.

Further, in exemplary embodiments the second guide 36 may be positioned at a location on the sheath 30 that corresponds to a location and/or orientation of the top wall 48 of the first guide 34. For example, the second guide 36 may be disposed on the top panel 80 of the sheath 30 in embodiments in which the top wall 48 of the first guide 34 is disposed adjacent to the top panel 80 at the proximal end 40 of the sheath 30. Alternatively, the second guide 36 may be disposed on the bottom panel 82 of the sheath 30 in embodiments in which the top wall 48 of the first guide 34 is disposed adjacent to the bottom panel 82 of the sheath 30. In still further exemplary embodiments in which the sheath 30 comprises a one-piece construction, the second guide 36 may be substantially radially aligned with the top wall 48 of the first guide 34 along the longitudinal axis 42. Due to the alignment of the first and second guides 34, 36, the user may pass the cuff connector 18 and the hose 28 through the first guide 34, and may pass at least the extension 24 through the passage 86 of the second guide 36 without rotating the cuff connector 18 or the hose 28 in a clockwise or counterclockwise manner relative to the longitudinal axis 42.

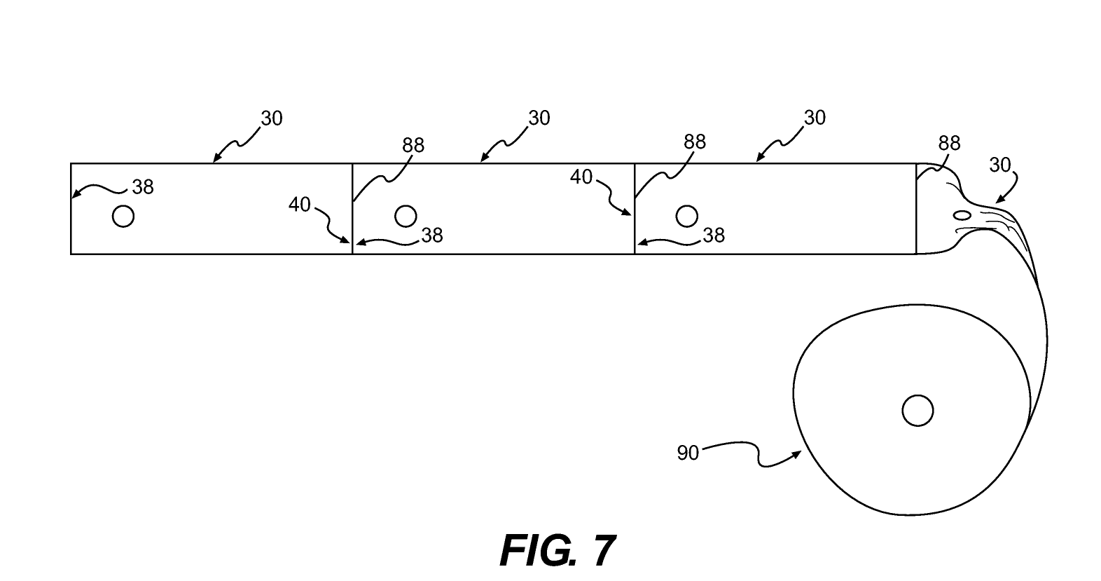

FIGS. 7-9 illustrate exemplary embodiments of the sheath 30 prior to use thereof. For example, as shown in FIG. 7, a plurality of sheaths 30 may be wound, coiled, and/or otherwise packaged in the form of a dispensable roll 90. In such an exemplary embodiment, the distal end 38 of each sheath 30 may be joined to the proximal end 40 of and adjacent sheath 30, and each of the sheaths 30 may be wound in a clockwise or counterclockwise manner to form the roll 90. In such an embodiment, the adjacent sheaths 30 may be joined in any conventional manner. For example, such adjacent sheaths 30 may be adhered or heat sealed along corresponding widths thereof. Alternatively, as shown in FIG. 7, adjacent sheaths 30 may be joined by a perforation 88 extending substantially along corresponding widths thereof. In this way, a single sheath 30 may be removed from the role 90, in a one-at-a-time manner, prior to use. It is understood that in further exemplary embodiments, the role 90 may be disposed within a box or other like container (not shown) to assist in removing sheaths 30 individually therefrom. It is understood that in the embodiment shown in FIG. 7 the guide 34 has been omitted for clarity. Nevertheless, it is understood that the first and second guides 34, 36 may be utilized with the sheaths 30 illustrated in FIG. 7 without departing from the scope of the present disclosure.

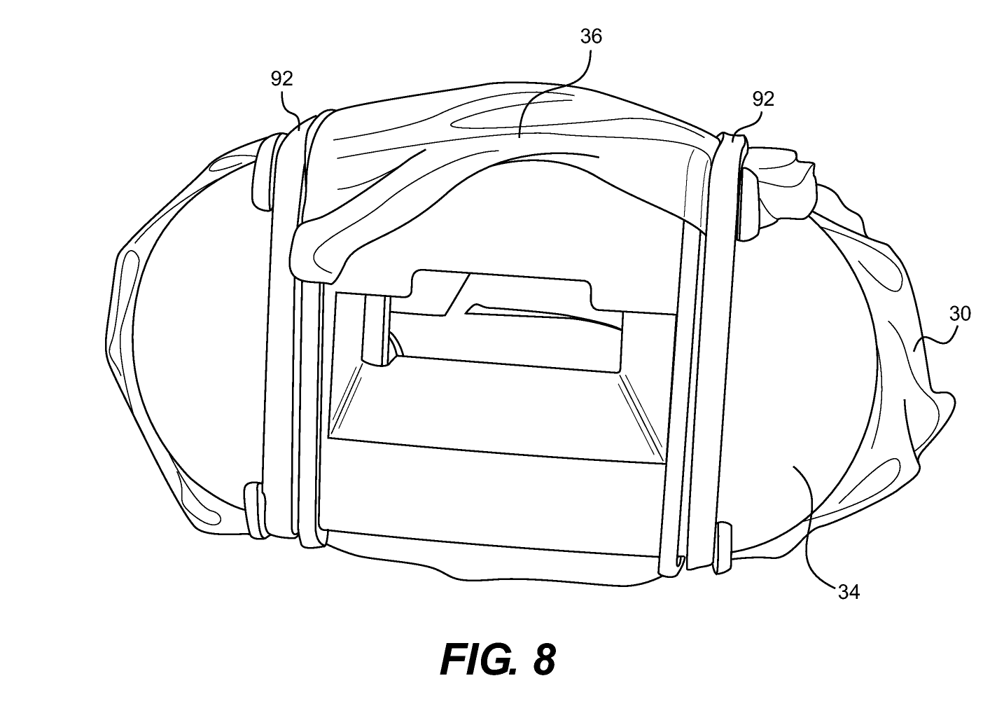

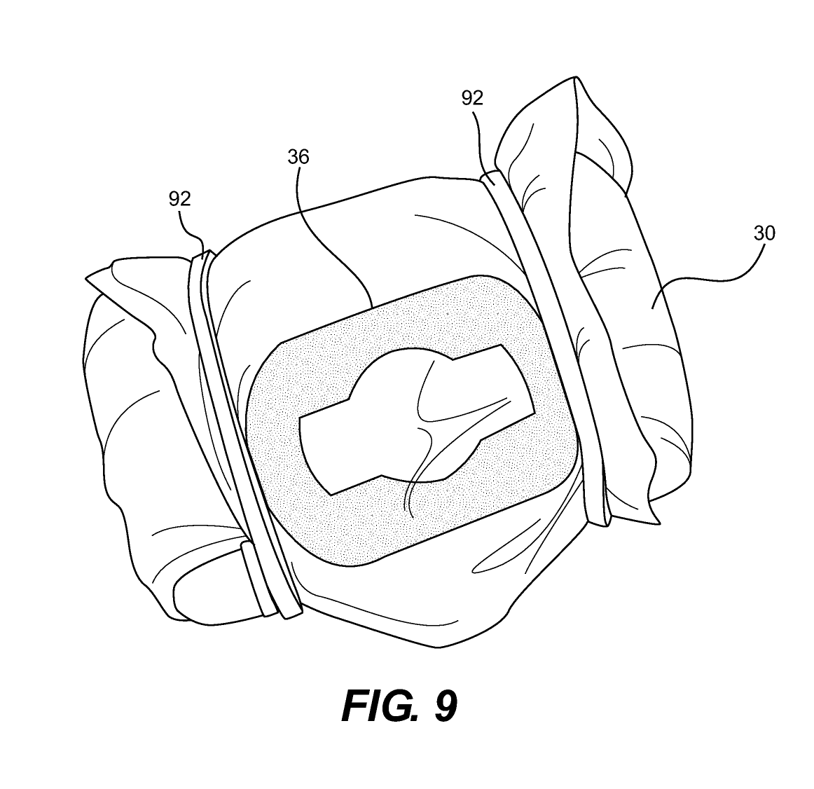

FIGS. 8 and 9, on the other hand, illustrate an exemplary embodiment in which each sheath 30 may be individually packaged in a tethered and/or otherwise compressed, individually packaged state. For example, each sheath 30 may be bound, wound, tied, banded, and/or otherwise tethered in the illustrated compressed state prior to use. Each sheath 30 may be bound by one or more tethers 92 comprising, for example, an elastic or inelastic band, tie, wrap, and/or other like binding component to retain each sheath 30 in the compressed state. For example, the sheath 30 may be wound in a clockwise or counterclockwise fashion about either the first guide 34 or the second guide 36, and one or more tethers 92 may be disposed about the wound sheath 30 to maintain the sheath 30 in such a compressed state. Accordingly, prior to use, the tether 92 may be removed, and the sheath may be at least partially unwound and/or otherwise decompressed to a substantially elongated, substantially extended state (such as the exemplary state shown in at least FIG. 2) such that the cuff connector 18 and at least a portion of the hose 28 may be inserted therein. In exemplary embodiments, once the tether 92 has been removed, the sheath 30 illustrated in FIGS. 8 and 9 may be partially unwound and/or otherwise partially decompressed. The user may then feed the cuff connector 18 and a minimal length of the hose 28 through the first guide 34 to the second guide. With the sheath 30 in such a partially decompressed state, the actual axial distance within the channel 44 along which the user must navigate the cuff connector 18 and the hose 28 to reach the second guide 36 may be advantageously minimized, thereby simplifying use of the sheath 30. For example, in such embodiments the axial distance within the channel 44 along which the user must navigate the cuff connector 18 and the hose 28 in order to reach the second guide 36 may be less than a corresponding distance associated with a sheath 30 that has been decompressed to a substantially elongated, substantially extended state. Once the cuff connector 18 reaches and/or engages the second guide 36 with the sheath 30 in a partially decompressed state, the sheath 30 may be extended along the hose 28 (i.e., away from the cuff connector 18 and the second guide 36, and in a direction opposite arrow 46) to achieve the substantially elongated, substantially extended state.

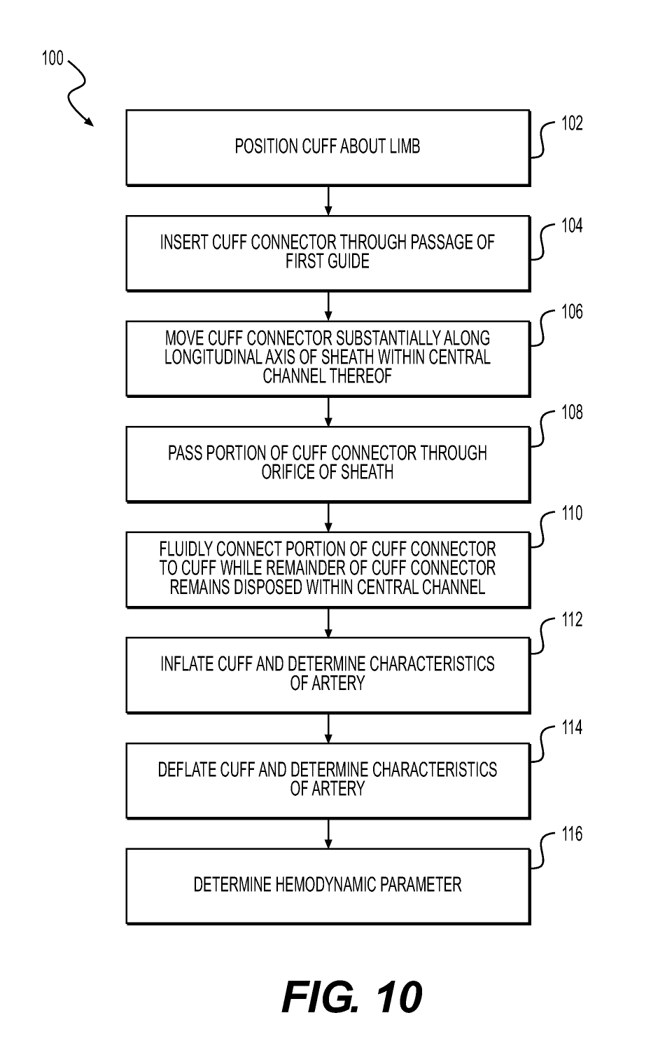

As illustrated by the flow chart 100 shown in FIG. 10, in exemplary embodiments, methods of monitoring the patient 14 and/or determining a hemodynamic parameter of the patient 14 may include determining one or more characteristics associated with the patient 14 and, in particular, one or more characteristics of the artery 22. Such methods may comprise oscillometric methods, auscultation methods, and/or any other known patient monitoring methods. For example, such methods may include positioning the cuff 12 about a limb 16 of the patient 14 (Step: 102). In exemplary embodiments, a substantially deflated cuff 12 may be positioned around a portion of an arm of patient 14, such as above the elbow (i.e., circumferentially around the bicep and tricep).

At an appropriate time, such as, for example, once the cuff 12 is properly positioned on the patient 14, the user may untether a sheath 30 from the compressed state shown in FIGS. 8 and 9. For example, the user may untether the sheath 30 by removing the one or more tethers 92 disposed about the sheath 30, and by unrolling the sheath 30 to the substantially extended position shown in FIG. 2 such that the cuff connector 18 is accessible. Alternatively, in exemplary embodiments in which adjacent sheaths 30 are wound on the role 90 shown in FIG. 7, the user may unwind a sheath 30 from the role 90 and detach the unwound sheath 30 from an adjacent sheath 30 by tearing the unwound sheath 30 at the perforation 88.

At Step: 104, the user may insert a cuff connector 18 through the passage 68 of the first guide 34. As the cuff connector 18 is inserted through the passage 68, the top wall 48, bottom wall 50, sidewalls 52, 54, 60, 62, and/or shoulders 56, 58 may guide movement of the cuff connector 18 in the first direction described above with respect to arrow 46. In particular, these surfaces of the passage 68 may guide movement of the cuff connector 18 in the first direction substantially parallel to the longitudinal axis 42 of the sheath 30. Likewise, these surfaces of the passage 68 may restrict movement of the cuff connector 18 in the one or more directions associated with arrows 70, 72, 74, 76. For example, the guide 34 may restrict movement of the cuff connector 18 in one or more directions substantially perpendicular to the first direction associated with arrow 46. As the cuff connector 118 is inserted through the passage 68 of the first guide 34 at Step: 104, the cuff connector 18 may move in sliding contact with these surfaces of the passage 68 to facilitate such guided and/or restricted movement. Additionally, at Step: 104, the guide 34 may restrict insertion of the cuff connector 18 into the passage 68 to a single unique orientation, such as the orientation illustrated in FIG. 4. In this way, the surfaces of the passage 68 may act as a keying feature that restricts orientation of the guide 34 to the single unique orientation described herein during movement through the passage 68.

At Step: 106, the user may move the cuff connector 18 substantially along the longitudinal axis 42 within the central channel 44 of the sheath 30. For example, the user may move the cuff connector 18 from the guide 34 at the proximal end 40 of the sheath 30 toward the distal end 38. As the cuff connector 18 is moved through the central channel 44, the user may maintain the cuff connector 18 at substantially the same unique orientation described above. As the cuff connector 18 approaches the proximal end 40, at least a portion of the hose 28 connected to the cuff connector 18 may extend through the passage 68 and into the central channel 44. It is understood, that the sheath 30 may form a substantially impervious barrier to contaminants carried by the hose 28 and/or the cuff connector 18 as the hose 28 and/or the cuff connector is disposed within the central channel 44. Accordingly, the sheath 30 may assist the user in connecting the cuff connector 18 with the cuff 12 without risking cross-contamination caused by the repeated use of a relatively unsanitary hose 28 and/or cuff connector 18.

At Step: 108, the user may pass a portion of the cuff connector 18 through the orifice 78 of the sheath 30 to facilitate connection with the cuff 12. For example, at Step: 108, the user may pass a portion of the cuff connector 18, such as the extension 24, through the orifice 78 in a direction substantially orthogonal to the first direction described above with respect to arrow 46. Such a substantially orthogonal direction may be illustrated by arrows 70, 72.

In exemplary embodiments, Step: 108 may include guiding movement of the cuff connector 18 with the second guide 36 overlaying the orifice 78. For example, at Step: 108, the user may guide movement of the cuff connector 18, with the second guide 36, through the orifice 78 in the second direction illustrated by arrow 70. In such exemplary embodiments, movement of the cuff connector 18 may be restricted by the second guide 36 in the first direction associated with arrow 46, and in one or more additional directions substantially perpendicular to the first direction. Such additional directions are illustrated by, for example, arrows 74, 76.

At Step: 110, the user may fluidly connect the portion of the cuff connector 18 passing through the orifice 78 to the cuff 12 while a remainder of the cuff connector 18 remains disposed within the central channel 44. For example, at Step: 110, the user may fluidly connect the extension 24 to one of the ports associated with the cuff 12. In exemplary embodiments, Step: 110 may further include connecting one or more of the retention components 22 to the port and/or other portions of the cuff 12 to facilitate a substantially fluid tight connection between the cuff 12 and the cuff connector 18.

At Step: 112, the cuff controller 32 may inflate the cuff 12 automatically (such as in accordance with a predetermined inflation protocol) or manually (such as in the case of a manually operated inflation bulb). The user may then determine one or more desired characteristics associated with the artery 22 while the cuff 12 is substantially inflated. It is understood that substantially inflating the cuff 12 at Step: 112 may substantially occlude the artery 22 such that substantially no (i.e., negligible) blood may flow through the artery 22. It is also understood that characteristics determined while the artery 22 is in such a substantially occluded state may include a systolic blood pressure, and such a blood pressure may be determined based on variations in the pressure within the cuff 12. For example, the cuff controller 32 may inflate the cuff 12 to an occlusion pressure that is greater than or equal to a systolic pressure of artery 22, and the user may measure and/or otherwise determine oscillations in cuff pressure according to one or more known oscillometric methods. In exemplary embodiments, the cuff controller 32 may utilize such information as inputs to one or more oscillometric pressure algorithms and may determine, for example, a systolic pressure associated with the artery 22 based on such information. Alternatively, once the cuff 12 is inflated to the occlusion pressure, the user may utilize known auscultation methods known in the art to determine a systolic pressure associated with the artery 22.

At Step: 114, the cuff controller 32 may deflate the cuff 12 automatically and/or manually similar to the inflation protocol described above. Additionally, at Step: 114 the user may determine one or more additional characteristics associated with artery 22 while the cuff 12 is substantially deflated. In an exemplary embodiment, the cuff controller 32 may deflate the cuff 12 to a deflated pressure less than the occlusion pressure, and at such a pressure, blood may resume flow through the artery 22. At such a deflation pressure, the artery 22 may be in a substantially unoccluded state, and as described above with respect to Step: 112, the user may measure and/or otherwise determine oscillations in cuff pressure according to one or more known oscillometric methods. The cuff controller 32 may utilize such information as inputs to one or more oscillometric pressure algorithms and may determine, for example, a diastolic pressure associated with artery 22 based on such information. Alternatively, once the cuff 12 is deflated to the deflation pressure (and/or during deflation), the user may utilize known auscultation methods known in the art to determine a diastolic pressure associated with the artery 22. It is understood that any of the characteristics determined at Step: 112 and Step: 114 may be stored in a memory of the cuff controller 32, and may be displayed and/or otherwise outputted by the cuff controller 32 as known in the art.

At Step: 116, the user and/or the cuff controller 32 may utilize one or more of the characteristics determined at Steps: 112 and 114 to calculate and/or otherwise determine a hemodynamic parameter of the patient 14. For example, information indicative of one or more such characteristics may be inputted into an algorithm, routine or program stored in memory of the cuff controller 32, and a processor associated therewith may determine an average blood pressure or other like hemodynamic parameter based on such characteristics.

Exemplary embodiments of the present disclosure may provide users with a sanitary means of using and reusing hoses, connectors, and other like components associated with inflating and deflating blood pressure cuff used for patient monitoring. For example, while the hose 28 and/or cuff connector 18 is disposed substantially within the sheath 30 of the present disclosure, the sheath 30 may form a substantially impervious barrier against air-borne pathogens, blood-borne pathogens, bacteria, viruses, and/or other harmful contaminants. As a result, the systems and methods described herein may provide improved protection against cross-contamination during repeated use of the hose 28 and/or cuff connector 18.

Other embodiments of the present disclosure will be apparent to those skilled in the art from consideration of the specification and practice of the disclosure contained herein. It is intended that the specification and examples be considered as exemplary only, with a true scope and spirit of the present disclosure being indicated by the following claims.

What is claimed is:

1. A cuff system, comprising:
a flexible substantially tubular sheath having a distal end, a proximal end, a central longitudinal axis, and a central channel extending along the longitudinal axis;
a first guide disposed at the proximal end of the sheath, the first guide configured to control insertion of a cuff connector into the central channel in a first direction substantially parallel to the longitudinal axis; and
an orifice proximate the distal end of the sheath and configured to enable fluid connection between a portion of the cuff connector and an inflatable cuff while a remainder of the cuff connector is disposed within the central channel.

2. The system of claim 1, wherein the sheath comprises a top panel and a bottom panel connected to the top panel, the central channel extending between the top panel and the bottom panel.

3. The system of claim 1, wherein the first guide restricts movement of the cuff connector in a second direction substantially orthogonal to the longitudinal axis.

4. The system of claim 1, wherein the first guide includes a top wall, and a bottom wall opposite and substantially parallel to the top wall, the top and bottom walls configured to guide movement of the cuff connector in the first direction.

5. The system of claim 4, wherein the top wall and the bottom wall each comprise substantially planar surfaces extending substantially parallel to the longitudinal axis.

6. The system of claim 4, wherein the first guide further comprises a first sidewall, and a second sidewall opposite and substantially parallel to the first sidewall, the first and second sidewalls extending substantially perpendicularly from the bottom wall and being configured to guide movement of the cuff connector in the first direction.

7. The system of claim 6, wherein the first guide further comprises a first shoulder extending substantially perpendicularly from the first sidewall and a second shoulder extending substantially perpendicularly from the second sidewall.

8. The system of claim 7, wherein the first guide further comprises a third sidewall extending substantially perpendicularly from the first shoulder and the top wall, and a fourth sidewall extending substantially perpendicularly from the second shoulder and the top wall.

9. The system of claim 1, further including a second guide proximate the distal end and configured to guide movement of the portion of the cuff connector through the orifice.

10. The system of claim 9, wherein the second guide is configured to guide movement of the portion of the cuff connector in a second direction substantially orthogonal to the longitudinal axis and the first direction.

11. The system of claim 9, wherein the second guide comprises a passage overlaying the orifice of the sheath.

12. The system of claim 9, wherein the second guide restricts movement of the portion in the first direction and in a third direction substantially perpendicular to the first direction.

13. The system of claim 1, wherein the first guide is configured to limit insertion of the cuff connector into the central channel in a single unique orientation relative to the sheath.

14. A cuff system, comprising:
a sheath including a distal end, a proximal end, a central longitudinal axis, and a central channel extending from the distal end to the proximal end along the longitudinal axis;
a first guide disposed at the proximal end of the sheath, the first guide including a passage configured to accept insertion of a cuff connector and to guide movement of the cuff connector through the first guide to the central channel in a first direction substantially parallel to the longitudinal axis; and
an orifice proximate the distal end of the sheath, the orifice permitting passage of a portion of the cuff connector therethrough in a second direction substantially orthogonal to the longitudinal axis.

15. The system of claim 14, wherein a portion of the sheath is substantially transparent such that the cuff connector is visible to a user of the system while the cuff connector is disposed within the central channel.

16. The system of claim 14, wherein the first guide includes a front face disposed outside of the central channel, and a back face opposite the front face, the passage extending from the front face to the back face.

17. The system of claim 14, further including a second guide having a passage overlaying the orifice of the sheath, the second guide configured to guide movement of the cuff connector in a second direction substantially orthogonal to the longitudinal axis.

18. The system of claim 17, wherein the second guide restricts movement of the cuff connector in the first direction and in a third direction substantially perpendicular to the first direction.

19. A method of monitoring a patient, comprising:
disposing an inflatable cuff about a limb of the patient;
inserting a cuff connector through a passage of a first guide, the first guide being disposed at a proximal end of a substantially tubular sheath and guiding movement of the cuff connector in a first direction substantially parallel to a longitudinal axis of the sheath;
moving the cuff connector substantially along the longitudinal axis, within a central channel of the sheath, from the proximal end toward a distal end of the sheath;
passing a portion of the cuff connector through an orifice of the sheath in a second direction substantially orthogonal to the first direction; and
fluidly connecting the portion to the cuff while a remainder of the cuff connector is disposed within the channel.

20. The method of claim 19, further including guiding movement of the cuff connector, with a second guide overlaying the orifice, through the orifice in the second direction.

21. The method of claim 20, further including restricting movement of the cuff connector, with the second guide, in the first direction and in a third direction substantially perpendicular to the first direction.

22. The method of claim 21, further including restricting movement of the cuff connector, with the first guide, in the second direction and in the third direction.

23. The method of claim 19, further including detaching the sheath from an additional substantially identical sheath via a perforation removably connecting the sheath to the additional sheath.

24. The method of claim 19, further including untethering the sheath, and transitioning the sheath from a substantially compressed state to a substantially extended state.

25. The method of claim 19, further including limiting, with the first guide, insertion of the cuff connector into the central channel in a single unique orientation relative to the sheath.

26. A method of manufacturing a sheath, comprising:
connecting a top panel of the sheath to a bottom panel of the sheath, the top and bottom panel forming a central channel 44 extending along a longitudinal axis of the sheath;
providing a first guide proximate a proximal end of the sheath, the first guide including a front face outside of the central channel, a back face opposite the front face and facing the channel, and a passage extending from the front face to the back face, the first guide being positioned such that the longitudinal axis extends substantially centrally through the passage; and
providing a second guide proximate a distal end of the sheath, the second guide overlaying an orifice of one of the top and bottom panels.

27. The method of claim 26, further including arranging the sheath in a compressed state wherein the sheath is wound about one of the first and second guides, and disposing a tether about the sheath in the compressed state.

28. The method of claim 26, further including joining the sheath to an additional adjacent sheath along a perforation extending substantially along corresponding widths of the respective sheaths, and winding the sheaths in the form of a dispensable roll.
